(19) United States
(12) Patent Application Publication
(10) Pub. No.: US 2021/0094955 A1
(43) Pub. Date: Apr. 1, 2021

(54) SALT AND CRYSTAL OF TRIAZOLOPYRAZINE DERIVATIVE

(71) Applicant: ASTELLAS PHARMA INC., Tokyo (JP)

(72) Inventors: Haruka KOJIMA, Tokyo (JP); Daisuke KAGA, Tokyo (JP); Hiroaki TANAKA, Tokyo (JP)

(21) Appl. No.: 16/755,799

(22) PCT Filed: Oct. 12, 2018
(86) PCT No.: PCT/JP2018/038017

§ 371 Date: Apr. 13, 2020

(30) Foreign Application Priority Data
Oct. 13, 2017 (JP) 2017-198985

(51) Int. Cl. C07D 487/04; C07C 62/04

(52) U.S. Cl. CPC C07D 487/04; C07B 2200/13; C07C 62/04

(57) ABSTRACT

[Problem] Provided are a salt of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone, and a crystal of the salt.

[Element for Solution] Salts of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone with various acids have been studied. As a result, a pharmaceutically acceptable salt of the compound and a crystal of the salt have been found.

That is, the present invention relates to a salt of the compound and a crystal of the salt. Furthermore, the present invention relates to a pharmaceutical composition including a salt of the compound or a crystal of the salt, and one or more excipients.

### TECHNICAL FIELD

The present invention relates to a novel salt of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (hereinafter, referred to as Compound A in some cases) and a crystal of the salt.

### BACKGROUND ART

It is known that Compound A has the chemical structure below, and is known to be useful as a neurokinin 3 (NK3) receptor antagonist against NK3 receptor related diseases such as depression, anxiety, psychosis, schizophrenia, psychotic disorders, bipolar disorder, cognitive disorder, Parkinson's disease, Alzheimer's disease, attention deficit hyperactivity disorder (ADHD), pain, convulsions, obesity, inflammatory disease such as irritable bowel syndrome (IBS) and inflammatory bowel disorder, emesis, pre-eclampsia, chronic obstructive pulmonary disease, asthma, airway-related diseases such as airway hyperreactivity, bronchoconstriction, and cough, urinary incontinence, reproduction disorders, contraception and sex hormone-dependent diseases (examples of the sex hormone-dependent disease include, but are not limited to, benign prostatic hyperplasia (BPH), prostatic hyperplasia, metastatic prostatic carcinoma, testicular cancer, breast cancer, ovarian cancer, androgen-dependent acne, male pattern baldness, endometriosis, abnormal puberty, uterine fibrosis, uterine fibroid, uterine leiomyoma, hormone-dependent cancer, hyperandrogenism, hirsutism, virilization, polycystic ovary syndrome (PCOS), premenstrual dysphoric disorder (PMDD), HAIR-AN syndrome (hyperandrogenism, insulin resistance, and acanthosis nigricans), ovarian hyperthecosis (HAIR-AN with hyperplasia of luteinized theca cells, in ovarian stroma), other manifestations of high intraovarian androgen concentrations (such as follicular maturation arrest, atresia, anovulation, dysmenorrhea, dysfunctional uterine bleeding, and infertility), androgen producing tumors (virilizing ovarian or adrenal tumors), menorrhagia, and adenomyosis), and hot flash (Patent Document 1 and Non-Patent Document 1). However, there is no specific disclosure of a pharmaceutically acceptable salt of Compound A and a crystal of the salt so far.

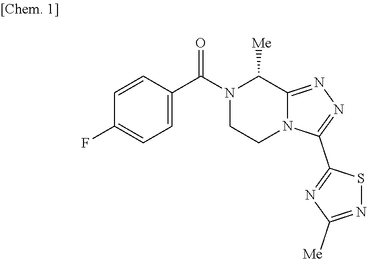 [SMILES: Cc1nsc(-c2nnc3n2CCN(C(=O)c2ccc(F)cc2)[C@@H]3C)n1]

### RELATED ART

### Patent Document

[Patent Document 1] WO 2014/154895

### Non-Patent Document

[Non-Patent Document 1] ACS Medicinal Chemistry Letters, 2015, 6, 736-740

### DISCLOSURE OF INVENTION

### Problems to be Solved by the Invention

In Patent Document 1 (Example 5) and Non-Patent Document 1 (Compound 3), Compound A is known as a free form, but a salt of Compound A and a crystal of the salt have not been found yet.

### Means for Solving the Problems

The present inventors have conducted studies about salts of compound A with various acids. As a result, a pharmaceutically acceptable salt of Compound A and a crystal of the salt have been found, thereby completing the present invention.

That is, the present invention relates to a novel salt of compound A and a crystal of the salt. Furthermore, the present invention relates to a pharmaceutical composition comprising a salt of Compound A or a crystal of the salt, and one or more excipients.

Further, the present invention relates to a salt of Compound A, which is an NK3 receptor antagonist; a salt of Compound A for use as an NK3 receptor antagonist; an NK3 receptor antagonist comprising a salt of Compound A; use of a salt of Compound A for the manufacture of a medicament or a pharmaceutical composition for treating a disease associated with an NK3 receptor; use of a salt of Compound A for treatment; a salt of Compound A for use in treatment of a disease associated with an NK3 receptor; and a method for treating a disease associated with an NK3 receptor, comprising administering to a subject an effective amount of a salt of Compound A. The “subject” is a human or other animal in need of prevention or treatment. In a certain embodiment, the subject is a human in need of the prevention or treatment.

### Effects of the Invention

The salt of Compound A and a crystal of the salt have NK3 receptor antagonistic activity and are expected to be useful as active ingredients of a pharmaceutical composition for preventing and/or treating NK3 receptor related diseases such as depression, anxiety, psychosis, schizophrenia, psychotic disorders, bipolar disorder, cognitive disorder, Parkinson's disease, Alzheimer's disease, attention deficit hyperactivity disorder (ADHD), pain, convulsions, obesity, inflammatory disease such as irritable bowel syndrome (IBS) and inflammatory bowel disorder, emesis, pre-eclampsia, chronic obstructive pulmonary disease, asthma, airway-related diseases such as airway hyperreactivity, bronchoconstriction, and cough, urinary incontinence, reproduction disorders, contraception and sex hormone-dependent diseases (examples of the sex hormone-dependent disease include, but are not limited to, benign prostatic hyperplasia (BPH), prostatic hyperplasia, metastatic prostatic carcinoma, testicular cancer, breast cancer, ovarian cancer, androgen-dependent acne, male pattern baldness, endometriosis, abnormal puberty, uterine fibrosis, uterine fibroid, uterine leiomyoma, hormone-dependent cancer, hyperandrogenism, hirsutism, virilization, polycystic ovary syndrome (PCOS), premenstrual dysphoric disorder (PMDD), HAIR-AN syndrome (hyperandrogenism, insulin resistance, and acanthosis nigricans), ovarian hyperthecosis (HAIR-AN with hyperplasia of luteinized theca cells, in ovarian stroma), other manifestations of high intraovarian androgen concentrations (such as follicular maturation arrest, atresia, anovulation, dysmenorrhea, dysfunctional uterine bleeding, and infertility), androgen producing tumors (virilizing ovarian or adrenal tumors), menorrhagia, and adenomyosis), and hot flash (including hot flash associated with pre-menopausal, menopausal, and post-menopausal conditions, and hot flash with hormonal therapy that lowers sex hormone levels, such as hot flash caused by treatment of breast cancer, uterine cancer, or prostate cancer).

### EMBODIMENTS FOR CARRYING OUT THE INVENTION

Hereinafter, the present invention will be described in detail.

In a case where a term used in the present specification is not specifically defined, the term is used in a sense generally accepted by those skilled in the art.

Some embodiments of the present invention are described below.

(1) A pharmaceutically acceptable salt of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone.
(2) The salt according to (1) above, wherein the salt is a hydrochloride, a hydrobromide, a sulfate, a p-toluenesulfonate, a methanesulfonate, or a benzenesulfonate.
(3) The salt according to (2) above, wherein the salt is the hydrochloride.
(4) The salt according to (2) above, wherein the salt is the hydrobromide.
(5) The salt according to (2) above, wherein the salt is the sulfate.
(6) The salt according to (2) above, wherein the salt is the p-toluenesulfonate.
(7) The salt according to (2) above, wherein the salt is the methanesulfonate.
(8) The salt according to (2) above, wherein the salt is the benzenesulfonate.
(9) A crystal of the salt according to (3) above.
(10) The crystal according to (9) above, wherein in a powder X-ray diffraction using Cu as a radiation source, the crystal has peaks at 2θ(°) of 6.4, 10.2, 12.0, 12.9, 14.5, 16.1, 16.8, 17.9, 22.7 and 26.7.
(11) A crystal of the salt according to (4) above.
(12) The crystal according to (11) above, wherein in a powder X-ray diffraction using Cu as a radiation source, the crystal has peaks at 2θ(°) of 6.4, 9.2, 10.3, 12.0, 12.8, 14.5, 17.0, 18.4, 22.6 and 26.1.
(13) A crystal of the salt according to (5) above.
(14) The crystal according to (13) above, wherein in a powder X-ray diffraction using Cu as a radiation source, the crystal has peaks at 2θ(°) of 6.2, 10.9, 12.5, 14.0, 15.3, 16.3, 18.7, 19.5, 21.3 and 24.1.
(15) The crystal according to (13) above, wherein in a powder X-ray diffraction using Cu as a radiation source, the crystal has peaks at 2θ(°) of 7.1, 10.0, 15.2, 16.7, 17.6, 18.5, 19.7, 23.5, 24.2, and 25.3.
(16) A crystal of the salt according to (6) above.
(17) The crystal according to (16) above wherein in a powder X-ray diffraction using Cu as a radiation source, the crystal has peaks at 2θ(°) of 6.7, 8.1, 10.0, 12.5, 13.0, 13.4, 14.9, 15.3, 18.0, and 23.5.
(18) A crystal of the salt according to (7) above.
(19) The crystal according to (18) above, wherein in a powder X-ray diffraction using Cu as a radiation source, the crystal has peaks at 2θ(°) of 7.0, 9.9, 15.1, 16.6, 17.5, 18.4, 19.3, 19.6, 23.2, and 24.0.
(20) A crystal of the salt according to (8) above.
(21) The crystal according to (20) above wherein in a powder X-ray diffraction using Cu as a radiation source, the crystal has peaks at 2θ(°) of 6.8, 8.2, 10.1, 11.6, 12.6, 14.9, 15.5, 18.1, 22.5, and 23.5.
(22) The salt according to (1) above, wherein the salt is 1-hydroxy-2-naphthoate.
(23) A crystal of the salt according to (22) above.
(24) The crystal according to (23) above, wherein in a powder X-ray diffraction using Cu as a radiation source, the crystal has peaks at 2θ(°) of 3.8, 6.4, 7.7, 8.5, 11.3, 12.9, 16.0, 16.7, 17.1, and 18.7.
(25) A pharmaceutical composition comprising the salt according to (1) above and a pharmaceutically acceptable excipient.
(26) The pharmaceutical composition according to (25) above, which is a pharmaceutical composition for treating a disease associated with an NK3 receptor.
(27) Use of the salt according to (1) above, which is for the manufacture of a pharmaceutical composition for treating a disease associated with an NK3 receptor.
(28) Use of the salt according to (1) above, which is for treating a disease associated with an NK3 receptor.
(29) The salt according to (1) above, for use in treating a disease associated with an NK3 receptor.
(30) A method for treating a disease associated with an NK3 receptor, the method comprising administering an effective amount of the salt according to (1) above to a subject.

Regarding a powder X-ray diffraction pattern described in this specification, crystal lattice spacing or an overall pattern is important in terms of nature of the data determining identity of crystals, while a diffraction angle and a diffraction intensity should not be interpreted strictly, since some errors may occur depending on a direction of crystal growth, a size of grains, and a measurement condition. In the present specification, a diffraction angle (2θ)(°) in the powder X-ray diffraction pattern is interpreted in consideration of an error range normally allowed in the measurement method, and may have an error range of ±0.2° in a certain embodiment. In addition, for example, in a case where measurement is performed in the state of a mixture with a pharmaceutical additive, in a peak present near a peak derived from the pharmaceutical additive and on a slope of a baseline, the peak may apparently shift by approximately ±0.3° in some cases.

The salt of Compound A is a pharmaceutically acceptable salt of Compound A, and refers to a salt of Compound A with a pharmaceutically acceptable acid. Specifically, examples of the salt include an acid addition salt with inorganic acids such as hydrochloric acid, hydrobromic acid, hydroiodic acid, sulfuric acid, nitric acid, and phosphoric acid, or organic acids such as formic acid, acetic acid, propionic acid, oxalic acid, malonic acid, succinic acid, fumaric acid, maleic acid, lactic acid, malic acid, mandelic acid, tartaric acid, dibenzoyltartaric acid, ditoluoyltartaric acid, citric acid, methanesulfonic acid, ethanesulfonic acid, benzenesulfonic acid, p-toluenesulfonic acid, aspartic acid, and glutamic acid. In addition, examples of a certain embodiment of the salt of Compound A include an acid addition salt with benzoic acid, 10-camphorsulfonic acid, chlorotheophylline, 1,2-ethanedisulfonic acid, gluceptin acid, gluconic acid, glucuronic acid, hippuric acid, isethionic acid, lactobionic acid, lauryl sulfate, methyl sulfate, naphthoic acid, napsylic acid, stearic acid, oleic acid, pamoic acid, polygalacturonic acid, sulfosalicylic acid, trifluoroacetic acid, 2,2-dichloroacetic acid, aceturic acid, adipic acid, alginic acid, ascorbic acid, 4-acetamidobenzoic acid, 2-(4-hydroxybenzoyl)benzoic acid, butyric acid, camphoric acid, capric acid, caproic acid, caprylic acid, carbonic acid, cinnamic acid, cyclamic acid, di(tert-butyl)naphthalenedisulfonic acid, di(tert-butyl)naphthalenesulfonic acid, 2-hydroxyethanesulfonic acid, galactic acid, gentisic acid, glucaric acid, glucoheptonic acid, glutaric acid, 2-oxoglutaric acid, glycerophosphate, glycolic acid, isobutyric acid, lauric acid, naphthalene-1,5-disulfonic acid, 1-hydroxy-2-naphthoic acid, nicotinic acid, orotic acid, palmitic acid, pyroglutamic acid, pyruvic acid, o-sulfobenzimide, salicylic acid, 4-aminosalicylic acid, sebacic acid, thiocyanic acid, and undecylenic acid.

In the present specification, a crystal of the salt of Compound A may include a co-crystal of Compound A and a pharmaceutically acceptable acid, in some cases. Specific examples thereof include co-crystal with the pharmaceutically acceptable acids described above.

Furthermore, the present invention also includes various hydrates and solvates of the salt of Compound A, and crystal polymorphic substances thereof.

In addition, the present invention also includes all pharmaceutically acceptable salts of Compound A labeled with one or more radioactive or non-radioactive isotopes.

The salt of Compound A can also be produced by subjecting Compound A to a conventional salt-forming reaction, and isolation and purification are performed by applying normal chemical operations such as extraction, fractional crystallization, and various types of fractional chromatography. In addition, Compound A can be produced by a known method or a modification thereof.

A pharmaceutical composition containing the salt of Compound A as an active ingredient can be prepared by using an excipient commonly used in the art, that is, by using a pharmaceutical excipient or a pharmaceutical carrier, by a method generally used.

Administration may be performed in any form of oral administration by a tablet, a pill, a capsule, granules, a powder, a liquid, or the like, or parenteral administration by injection for intraarticular, intravenous, or intramuscular, a suppository, eye drops, an eye ointment, a transdermal liquid preparations, an ointment, a transdermal patch, a transmucosal liquid preparations, a transmucosal patch, inhalation, or the like.

As a solid composition for the oral administration, a tablet, a powder, granules and the like are used. In such a solid composition, one or more active ingredients are mixed with at least one inert excipient. The composition may contain an inert additive such as a lubricant, a disintegrant, a stabilizer, and a solubilizing agent in accordance with a conventional method. The tablet, the powder, the granules, or the pill may be coated with wax or a film made of sugar coating or gastric or enteric substance, as needed.

A liquid composition for the oral administration include an emulsion, a solution, a suspension, a syrup, an elixir, or the like which is pharmaceutically acceptable, and include a commonly used inert diluent such as purified water or ethanol. The liquid composition may contain an auxiliary such as a solubilizing agent, a wetting agent, and a suspension, a sweetening agent, a flavor, an aromatic substance, and a preservative, in addition to the inert diluent.

The injection for the parenteral administration contains a sterile aqueous or non-aqueous solution, a suspension, or an emulsion. Examples of an aqueous solvent include distilled water for injection or physiological saline. Examples of a non-aqueous solvent include alcohol such as ethanol. Such a composition may further include a tonicity agent, a preservative, a wetting agent, an emulsifier, a dispersant, a stabilizer, or a solubilizing agent. These are sterilized by, for example, filtration through a bacteria retaining filter, blending of a bactericide or irradiation. In addition, these can be used by preparing a sterile solid composition, dissolving or suspending in sterile water or a sterile injection solvent before use.

External preparations include an ointment, a plaster, cream, jellies, a cataplasm, a spray, lotions, eye drops, an eye ointment, and the like. The external preparations contain an ointment base, a lotion base, an aqueous or non-aqueous liquid preparation, a suspension, an emulsion, and the like, which are commonly used.

A transmucosal preparation such as an inhalants or nasal preparation is used in a form of solid, liquid, or semi-solid, and can be produced in accordance with a known method of related art. For example, a known excipient, and a pH adjuster, a preservative, a surfactant, a lubricant, a stabilizer, a thickener, or the like may further be added thereto, as appropriate. For administration, an appropriate inhalation or insufflation device can be used. For example, a compound can be administered alone, as a powder in a formulated mixture, or as a solution or suspension in combination with a pharmaceutically acceptable carrier, using a known device or a nebulizer, such as metered dose inhalation device. A dry powder inhaler or the like may be for single or multiple administrations and a dry powder or a powder-containing capsule can be used. Alternatively, a form of a pressurized aerosol spray using a suitable propellant agent, for example, a suitable gas such as chlorofluoroalkane or carbon dioxide may be used.

In the case of normal oral administration, a daily dosage is approximately 0.001 to 100 mg/kg, preferably 0.1 to 30 mg/kg, more preferably 0.1 to 10 mg/kg, per body weight, and is administered once or in 2 to 4 divided doses. In a case of intravenous administration, a daily dosage is appropriately approximately 0.0001 to 10 mg/kg, per body weight, and is administered once or in several divided doses a day. In addition, in a case of a transmucosal preparation, approximately 0.001 to 100 mg/kg per body weight is administered once or in several divided doses a day. The dosage is appropriately determined according to individual cases in consideration of symptoms, age, sex, and the like.

It varies depending on a route of administration, a dosage form, a site of administration, a type of an excipient or an additive, the pharmaceutical composition of the present invention contains one or more salts of Compound A, which are active ingredients in 0.01% to 100% by weight, and in a certain embodiment, 0.01% to 50% by weight.

The salt of Compound A can be used in combination with various agents for treating or preventing diseases on which Compound A is considered to be effective. In the combination use, the administration may be performed simultaneously, performed separately and continuously, or performed at desired time intervals. Preparations for simultaneous administration may be combined preparations or separately prepared.

### TEST EXAMPLE

Test methods, measurement conditions, and results of a solubility test of Compound A and various salts of Compound A are shown below.

### Test Method 1: Test Method of Free Form, Hydrochloride, Hydrobromide, Sulfate, Methanesulfonate, and Benzenesulfonate

Into a 10 mL centrifuge tube, 0.5 to 1 mg of a sample was weighed and each dissolving solution (see below) was added thereto to be approximately 150 μg/mL. In order to uniformly disperse the sample in the dissolving solution, the sample was irradiated with ultrasonic waves in a water tank for a short time, and then vigorously shaken at room temperature for 10 minutes using a shaker SA-31 manufactured by Yamato Scientific Co., Ltd. The solution after shaken was filtered with a filter, and a concentrations of the compound in filtrate was quantified by HPLC. The following was used as the dissolving solution.

JP1 (Japanese Pharmacopoeia 1^{st} Fluid for disintegration test): manufactured by Nacalai Tesque Inc.

JP2 (Japanese Pharmacopoeia 2^{nd} Fluid for disintegration test): manufactured by Nacalai Tesque Inc.

JP2+TC: Add sodium taurocholate to Japanese Pharmacopoeia 2^{nd} Fluid for disintegration test to obtain a concentration of 15 mM.

FaSSIF (artificial intestinal fluid (fasted state)): To 1 L of water, 3 mmol of sodium taurocholate, 0.75 mmol of lecithin, 3.9 g of potassium dihydrogen phosphate, and 7.7 g of potassium chloride are added, and a pH thereof is adjusted to approximately 6.5 using sodium hydroxide.

FeSSIF (artificial intestinal fluid (fed state)): To 1 L of water, 15 mmol of sodium taurocholate, 3.75 mmol of lecithin, 8.65 g of acetic acid, and 15.2 g of potassium chloride are added, and a pH is adjusted to approximately 5.0 using sodium hydroxide.

### Test Method 2: Test Method for p-Toluenesulfonate and 1-Hydroxy-2-Naphthoate

Into a 10 mL centrifuge tube, 1 to 1.3 mg of a sample was weighed, and each dissolving solution (see below) was added thereto to be approximately 300 μg/mL. In order to uniformly disperse the sample in the dissolving solution, the sample was irradiated with ultrasonic waves in a water tank for a short time, and then vigorously shaken at room temperature for 10 minutes using a shaker SA-31 manufactured by Yamato Scientific Co., Ltd. The solution after shaken was filtered with a filter, and a concentration of the compound in filtrate was quantified by HPLC. The same dissolving solution as in Test Method 1 was used.

(Measurement condition 1 using HPLC: Measurement conditions of free form, hydrochloride, hydrobromide, sulfate, methanesulfonate, and benzenesulfonate)

Equipment: LC-1260 manufactured by Agilent Technologies

Mobile phase: A: 0.01 M aqueous solution of ammonium acetate, B: 0.01 M methanol solution of ammonium acetate

Flow rate: 0.3 mL/min

Column: Develosil Combi-RP-5 5 μm (2.0 mm×50 mm)

Column temperature: 40° C.

Gradient conditions: proportion of mobile phase B 10-50% (0-0.5 min), 50-100% (0.5-5 min), 100% (5-7 min), 10% (7.1-11.5 min)

Injection volume: 10 μL

Detection wavelength: 254 nm

(Measurement condition 2 using HPLC: Measurement conditions of p-toluenesulfonate and 1-hydroxy-2-naphthoate)

Equipment: LC-1260 manufactured by Agilent Technologies

Mobile phase: A: 0.1% aqueous perchloric acid, B: methanol

Flow rate: 0.3 mL/min

Column: YMC-Triart C18 S-3 μm (2.0 mm×50 mm)

Column temperature: 40° C.

Gradient conditions: proportion of mobile phase B 10-50% (0-0.5 min), 50-100% (0.5-5 min), 100% (5-7 min), 10% (7.1-11.5 min)

Injection volume: 10 μL

Detection wavelength: 254 nm

(Result)

Table 1 shows results of solubility of Compound A and various salts of Compound A in terms of their free forms. Ex indicates Example Number to be described later.

TABLE 1

| Ex | Solubility (μg/mL) | JP1 | JP2 | JP2 + TC | FaSSIF | FeSSIF |
|---|---|---|---|---|---|---|
| | Compound A | 66.6 | 65.4 | 82.9 | 36.5 | 31.9 |
| 1 | Hydrochloride of Compound A | >100 | >100 | >100 | >100 | >100 |
| 2 | Hydrobromide of Compound A | >100 | >100 | >100 | >100 | >100 |
| 3 | Sulfate of Compound A | >100 | >100 | >100 | >100 | >100 |
| 4 | Sulfate of Compound A | >100 | >100 | >100 | >100 | >100 |
| 5 | p-Toluenesulfonate of Compound A | >100 | >100 | >100 | >100 | >100 |
| 6 | Methanesulfonate of Compound A | >100 | >100 | >100 | >100 | >100 |
| 7 | Benzenesulfonate of Compound A | >100 | >100 | >100 | >100 | >100 |
| 8 | 1-Hydroxy-2-naphthoate of | 58.9 | >100 | >100 | >100 | >100 |
| | Compound A | | | | | |

### EXAMPLE

Hereinafter, the present invention will be described specifically with reference to Examples. However, the present invention is not limited by these Examples and does not limit the scope of the present invention.

In addition, in Examples, the following abbreviations may be used.

ESI+: m/z value in mass spectrometry (ionization method ESI, unless otherwise noted, [M+H]⁺), NMR (DMSO-d6): δ (ppm) of a peak in ¹H-NMR in DMSO-d₆), s: single line (spectrum), d: double line (spectrum), m: multiple line (spectrum), br: broad line (spectrum). In addition, br s indicates a wide single line, br d indicates a wide double line, and ddd indicates three double lines having different J values.

The measurement of powder X-ray diffraction was carried out using RINT-TTRII, under conditions of tube: Cu, tube current: 300 mA, tube voltage: 50 kV, sampling width: 0.020°, scanning speed: 4°/min, wavelength: 1.5418 Λ, and diffraction angle range to be measured (2θ): 2.5° to 40°. Handling of the apparatus including data processing followed the method and procedure instructed by each apparatus.

In addition, for convenience, a concentration mol/L is represented as M. For example, a 1 M aqueous sodium hydroxide solution refers to a 1 mol/L of aqueous sodium hydroxide solution.

### Reference Example 1 Production of Compound A

It was produced according to a method described in WO 2014/154895.

### Example 1

A mixture of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (100 mg) and ethyl acetate (3 mL) was stirred at 60° C. to be dissolved, and hydrogen chloride (4 M ethyl acetate solution, 84 μL) was added thereto, and stirred at room temperature for 15 hours. A precipitated solid was collected by filtration and dried under reduced pressure to obtain crystals (109 mg) of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone hydrochloride.

NMR(DMSO-d6): 1.62 (3H, d, J=6.8 Hz), 2.70 (3H, s), 3.59-3.71 (1H, m), 4.09-4.37 (3H, m), 4.65-4.72 (1H, m), 5.23-5.90 (1H, m), 7.30-7.36 (2H, m), 7.58-7.62 (2H, m)

ESI+: 359

Elemental analysis (%) (values in parentheses indicate theoretical values): C: 48.75 (48.67), H: 4.10 (4.08), N: 21.22 (21.28), S: 8.20 (8.12), 8.64 (8.98), F: 4.80 (4.81)

The crystals obtained in Example 1 have peaks at 2θ(°) of 6.4, 10.2, 12.0, 12.9, 14.5, 16.1, 16.8, 17.9, 22.7, and 26.7 in the powder X-ray diffraction.

### Example 2

A mixture of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (200 mg) and 2-propanol (3 mL) was stirred at 75° C. to be dissolved, and hydrobromic acid (47%, 77 μL) was added thereto, and stirred at room temperature for 15 hours. A precipitated solid was collected by filtration and dried under reduced pressure to obtain crystals (214 mg) of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone hydrobromide.

NMR(DMSO-d6): 1.62 (3H, d, J=6.8 Hz), 2.70 (3H, s), 3.59-3.70 (1H, m), 3.89-4.17 (1H, m), 4.26-4.35 (1H, m), 4.65-4.72 (1H, m), 4.77-5.16 (1H, m), 5.57-5.95 (1H, m), 7.31-7.36 (2H, m), 7.58-7.62 (2H, m)

ESI+: 359

Elemental analysis (%) (values in parentheses indicate theoretical values): C: 43.93 (43.74), H: 3.74 (3.67), N: 19.23 (19.13), S: 7.31 (7.30), Br: 17.94 (18.19), F: 4.29 (4.32)

The crystals obtained in Example 2 have peaks at 2θ(°) of 6.4, 9.2, 10.3, 12.0, 12.8, 14.5, 17.0, 18.4, 22.6, and 26.1 in the powder X-ray diffraction.

### Example 3

A concentrated sulfuric acid (8.2 μL) was added to a mixture of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (100 mg) and 2-butanone (3 mL), and stirred at room temperature for 15 hours. A precipitated solid was collected by filtration and dried under reduced pressure to obtain crystals (62 mg) of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone sulfate.

NMR(DMSO-d6): 1.62 (3H, d, J=6.8 Hz), 2.70 (3H, s), 3.59-3.70 (1H, m), 3.91-4.20 (1H, m), 4.26-4.36 (1H, m), 4.65-4.72 (1H, m), 5.54-5.88 (1H, m), 7.22-7.50 (4H, m), 7.58-7.64 (2H, m)

ESI+: 359

Elemental analysis (%) (values in parentheses indicate theoretical values): C: 42.25 (42.10), H: 3.79 (3.75), N: 18.32 (18.41), S: 13.90 (14.05), F: 4.02 (4.16)

The crystals obtained in Example 3 have peaks at 2θ(°) of 6.2, 10.9, 12.5, 14.0, 15.3, 16.3, 18.7, 19.5, 21.3, and 24.1 in the powder X-ray diffraction.

### Example 4

A concentrated sulfuric acid (33 μL) was added to a mixture of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (200 mg) and 2-butanone (2 mL), and stirred at room temperature for 15 hours. A precipitated solid was collected by filtration and dried under reduced pressure to obtain crystals (212 mg) of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone sulfate.

NMR(DMSO-d6): 1.62 (3H, d, J=6.8 Hz), 2.70 (3H, s), 3.59-3.70 (1H, m), 3.84-4.16 (1H, m), 4.25-4.35 (1H, m), 4.65-4.77 (1H, m), 5.54-5.90 (1H, m), 7.18-7.42 (4H, m), 7.57-7.62 (2H, m)

ESI+: 359

Elemental analysis (%) (values in parentheses indicate theoretical values): C: 42.22 (42.10), H: 3.84 (3.75), N: 18.43 (18.41), S: 14.04 (14.05), F: 4.15 (4.16) The crystals obtained in Example 4 have peaks at 2θ(°) of 7.1, 10.0, 15.2, 16.7, 17.6, 18.5, 19.7, 23.5, 24.2, and 25.3 in the powder X-ray diffraction.

### Example 5

p-Toluenesulfonic acid monohydrate (64 mg) was added to a mixture of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (100 mg) and ethyl acetate (4 mL), and stirred at 60° C. for 5 minutes, and then stirred at room temperature for 15 hours. A precipitated solid was collected by filtration and dried under reduced pressure to obtain crystals (140 mg) of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone p-toluenesulfonate.

NMR(DMSO-d6): 1.62 (3H, d, J=6.8 Hz), 2.28 (3H, s), 2.69 (3H, s), 3.57-3.69 (1H, m), 3.88-4.17 (1H, m), 4.25-4.36 (1H, m), 4.64-4.72 (1H, m) 4.81-5.14 (1H, m), 5.55-5.88 (1H, m), 7.11 (2H, d, J=8.0 Hz), 7.30-7.36 (2H, m), 7.47 (2H, d, J=8.0 Hz), 7.57-7.62 (2H, m)

ESI+: 359

Elemental analysis (%) (values in parentheses indicate theoretical values): C: 51.83 (52.06), H: 4.21 (4.37), N: 15.65 (15.84), S: 12.16 (12.09), F: 3.55 (3.58)

The crystals obtained in Example 5 have peaks at 2θ(°) of 6.7, 8.1, 10.0, 12.5, 13.0, 13.4, 14.9, 15.3, 18.0, and 23.5 in the powder X-ray diffraction.

### Example 6

Methanesulfonic acid (22 μL) was added to a mixture of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (100 mg) and ethyl acetate (4 mL), and stirred at 60° C. for 5 minutes, and then stirred at room temperature for 15 hours. A precipitated solid was collected by filtration and dried under reduced pressure to obtain crystals (110 mg) of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone methanesulfonate.

NMR(DMSO-d6): 1.63 (3H, d, J=6.8 Hz), 2.38 (3H, s), 2.70 (3H, s), 3.60-3.71 (1H, m), 3.90-4.16 (1H, m), 4.26-4.36 (1H, m), 4.65-4.73 (1H, m), 5.23-5.92 (2H, m), 7.31-7.37 (2H, m), 7.58-7.63 (2H, m)

ESI+: 359

Elemental analysis (%) (values in parentheses indicate theoretical values): C: 44.86 (44.92), H: 4.10 (4.21), N: 18.51 (18.49), S: 14.42 (14.11), F: 4.17 (4.18)

The crystals obtained in Example 6 have peaks at 2θ(°) of 7.0, 9.9, 15.1, 16.6, 17.5, 18.4, 19.3, 19.6, 23.2, and 24.0 in the powder X-ray diffraction.

### Example 7

Benzenesulfonic acid monohydrate (59 mg) was added to a mixture of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (100 mg) and ethyl acetate (4 mL), and stirred at 60° C. for 5 minutes, and then stirred at room temperature for 15 hours. A precipitated solid was collected by filtration and dried under reduced pressure to obtain crystals (106 mg) of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone benzenesulfonate.

NMR(DMSO-d6): 1.62 (3H, d, J=6.8 Hz), 2.69 (3H, s), 3.59-3.69 (1H, m), 3.88-4.17 (1H, m), 4.26-4.34 (1H, m), 4.52-4.80 (2H, m), 5.57-5.88 (1H, m), 7.27-7.35 (5H, m), 7.57-7.62 (4H, m)

ESI+: 359

Elemental analysis (%) (values in parentheses indicate theoretical values): C: 50.85 (51.15), H: 4.07 (4.10), N: 16.22 (16.27), S: 12.58 (12.41), F: 3.67 (3.68)

The crystals obtained in Example 7 have peaks at 2θ(°) of 6.8, 8.2, 10.1, 11.6, 12.6, 14.9, 15.5, 18.1, 22.5, and 23.5 in the powder X-ray diffraction.

### Example 8

Methanol (1.5 mL) was added to a mixture of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone (312 mg) and 1-hydroxy-2-naphthoic acid (173 mg), and the mixture was heated to 60° C. (bath temperature) to be dissolved, and then stirred at room temperature overnight. A precipitated solid was collected by filtration and dried under reduced pressure to obtain crystals (210 mg) of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone 1-hydroxy-2-naphthoate.

NMR (DMSO-d6): 1.62 (3H, d, J=6.9 Hz), 2.69 (3H, s), 3.57-3.73 (1H, m), 3.85-4.21 (1H, m), 4.24-4.40 (1H, m), 4.60-4.76 (1H, m), 5.54-5.95 (1H, m), 7.30-7.36 (2H, m), 7.40 (1H, d, J=8.6 Hz), 7.57-7.63 (3H, m), 7.68 (1H, ddd, J=8.2, 6.9, 1.5 Hz), 7.75 (1H, d, J=8.9 Hz), 7.91 (1H, br d, J=8.1 Hz), 8.27-8.31 (1H, m), 12.70 (1H, br s), 14.06 (1H, br s)

ESI+: 359

Elemental analysis (%) (values in parentheses indicate theoretical values): C: 59.44 (59.33), H: 4.32 (4.24), N: 15.28 (15.38), S: 5.95 (5.87), F: 3.46 (3.48) The crystals obtained in Example 8 have peaks at 2θ(°) of 3.8, 6.4, 7.7, 8.5, 11.3, 12.9, 16.0, 16.7, 17.1, and 18.7 in the powder X-ray diffraction.

### INDUSTRIAL APPLICABILITY

The present invention can provide a novel salt of Compound A and a crystal of the salt. The salt of the Compound A and the crystal of the salt are expected to be useful as active ingredients of a pharmaceutical composition for preventing and/or treating NK3 receptor related diseases such as depression, anxiety, psychosis, schizophrenia, psychotic disorders, bipolar disorder, cognitive disorder, Parkinson's disease, Alzheimer's disease, attention deficit hyperactivity disorder (ADHD), pain, convulsions, obesity, inflammatory disease such as irritable bowel syndrome (IBS) and inflammatory bowel disorder, emesis, pre-eclampsia, chronic obstructive pulmonary disease, asthma, airway-related diseases such as airway hyperreactivity, bronchoconstriction, and cough, urinary incontinence, reproduction disorders, contraception and sex hormone-dependent diseases (examples of the sex hormone-dependent disease include, but are not limited to, benign prostatic hyperplasia (BPH), prostatic hyperplasia, metastatic prostatic carcinoma, testicular cancer, breast cancer, ovarian cancer, androgen-dependent acne, male pattern baldness, endometriosis, abnormal puberty, uterine fibrosis, uterine fibroid, uterine leiomyoma, hormone-dependent cancer, hyperandrogenism, hirsutism, virilization, polycystic ovary syndrome (PCOS), premenstrual dysphoric disorder (PMDD), HAIR-AN syndrome (hyperandrogenism, insulin resistance, and acanthosis nigricans), ovarian hyperthecosis (HAIR-AN with hyperplasia of luteinized theca cells, in ovarian stroma), other manifestations of high intraovarian androgen concentrations (such as follicular maturation arrest, atresia, anovulation, dysmenorrhea, dysfunctional uterine bleeding, and infertility), androgen producing tumors (virilizing ovarian or adrenal tumors), menorrhagia, and adenomyosis), and hot flash (including hot flashes associated with pre-menopausal, menopausal, and post-menopausal conditions, and hot flashes with hormonal therapy that lowers sex hormone levels, such as hot flashes caused by treatment of breast cancer, uterine cancer, or prostate cancer).

1. A pharmaceutically acceptable salt of (4-fluorophenyl)[(8R)-8-methyl-3-(3-methyl-1,2,4-thiadiazol-5-yl)-5,6-dihydro[1,2,4]triazolo[4,3-a]pyrazin-7(8H)-yl]methanone.

2. The salt according to claim 1, wherein the salt is a hydrochloride, a hydrobromide, a sulfate, a p-toluenesulfonate, a methanesulfonate, or a benzenesulfonate.

3. The salt according to claim 2, wherein the salt is the hydrochloride.

4. The salt according to claim 2, wherein the salt is the hydrobromide.

5. The salt according to claim 2, wherein the salt is the sulfate.

6. The salt according to claim 2, wherein the salt is the p-toluenesulfonate.

7. The salt according to claim 2, wherein the salt is the methanesulfonate.

8. The salt according to claim 2, wherein the salt is the benzenesulfonate.

9. A crystal of the salt according to claim 3.

10. The crystal according to claim 9, wherein in a powder X-ray diffraction using Cu as a radiation source, the crystal has peaks at 2θ(°) of 6.4, 10.2, 12.0, 12.9, 14.5, 16.1, 16.8, 17.9, 22.7 and 26.7.

11. A crystal of the salt according to claim 4.

12. The crystal according to claim 11, wherein in a powder X-ray diffraction using Cu as a radiation source, the crystal has peaks at 2θ(°) of 6.4, 9.2, 10.3, 12.0, 12.8, 14.5, 17.0, 18.4, 22.6 and 26.1.

13. A crystal of the salt according to claim 5.

14. The crystal according to claim 13, wherein in a powder X-ray diffraction using Cu as a radiation source, the crystal has peaks at 2θ(°) of 6.2, 10.9, 12.5, 14.0, 15.3, 16.3, 18.7, 19.5, 21.3 and 24.1.

15. The crystal according to claim 13, wherein in a powder X-ray diffraction using Cu as a radiation source, the crystal has peaks at 2θ(°) of 7.1, 10.0, 15.2, 16.7, 17.6, 18.5, 19.7, 23.5, 24.2, and 25.3.

16. A crystal of the salt according to claim 6.

17. The crystal according to claim 16, wherein in a powder X-ray diffraction using Cu as a radiation source, the crystal has peaks at 2θ(°) of 6.7, 8.1, 10.0, 12.5, 13.0, 13.4, 14.9, 15.3, 18.0, and 23.5.

18. A crystal of the salt according to claim 7.

19. The crystal according to claim 18, wherein in a powder X-ray diffraction using Cu as a radiation source, the crystal has peaks at 2θ(°) of 7.0, 9.9, 15.1, 16.6, 17.5, 18.4, 19.3, 19.6, 23.2, and 24.0.

20. A crystal of the salt according to claim 8.

21. The crystal according to claim 20, wherein in a powder X-ray diffraction using Cu as a radiation source, the crystal has peaks at 2θ(°) of 6.8, 8.2, 10.1, 11.6, 12.6, 14.9, 15.5, 18.1, 22.5, and 23.5.

22. The salt according to claim 1, wherein the salt is 1-hydroxy-2-naphthoate.

23. A crystal of the salt according to claim 22.

24. The crystal according to claim 23, wherein in a powder X-ray diffraction using Cu as a radiation source, the crystal has peaks at 2θ(°) of 3.8, 6.4, 7.7, 8.5, 11.3, 12.9, 16.0, 16.7, 17.1, and 18.7.

25. A pharmaceutical composition comprising the salt according to claim 1 and a pharmaceutically acceptable excipient.

26. The pharmaceutical composition according to claim 25, which is a pharmaceutical composition for treating a disease associated with an NK3 receptor.

27. A method for the manufacture of a pharmaceutical composition for treating a disease associated with an NK3 receptor comprising providing the salt of claim 1, and combining the salt with a pharmaceutically acceptable excipient.

28. (canceled)

29. The salt according to claim 1, for use in treating a disease associated with an NK3 receptor.

30. A method for treating a disease associated with an NK3 receptor, the method comprising administering an effective amount of the salt according to claim 1 to a subject.
